(19) United States
(12) Patent Application Publication
(10) Pub. No.: US 2006/0233837 A1
(43) Pub. Date: Oct. 19, 2006

(54) Vibrio cholerae vaccine candidates, the methods of their constructing and medicinal preparations derived thereof

(76) Inventors: Javier Campos Gomez, Ciudad Habana (CU); Rafael Alfredo Fando Calzada, Ciudad Habana (CU); Boris Luis Rodriguez Gonzalez, Ciudad Habana (CU); Edgar Valle Diaz, Camaguey (CU); Talena Yamile Ledon Perez, Ciuda Hababa (CU); Anisia Juana Silva Cabrera, Atlanta, GA (US); Jorge Antonio Benitez Robles, Atlanta, GA (US); Tomas Marcelino Moreira Hernandez, Ciudad Habana (CU)

Correspondence Address: LACKENBACH SIEGEL, LLP, LACKENBACH SIEGEL BUILDING, 1 CHASE ROAD, SCARSDALE, NY, 10583, US

(21) Appl. No.: 11/108,922

(22) Filed: Apr. 19, 2005

(51) Int. Cl. A61K 39/106

(52) U.S. Cl. 424/261.1

(57) ABSTRACT

A single oral administration dose medicinal preparation is derived from Vibrio cholerae vaccine strains which have a disrupted hemagglutinin protease gene and which are tagged with celA coding functions from Clostridium thermocellum are described. The strains are freeze-dried and derived from non toxigenic parented strains. The medicinal preparation includes lyopectrants. The single dose of 10⁷ to 10⁹ viable cells/single dose provides effective immunization, with antibacterial antibodies present in about 1 day to a peak activity in about 14 days.

### TECHNICAL SECTOR

Biotechnology is the field of this invention, more specifically the methods to construct the pharmaceutically active ingredient of a cholera vaccine as well as the methods to prepare and the composition of the medicinal preparation suitable for oral administration to induce immunological protection against cholera.

### BACKGROUND OF THE INVENTION

Definitions:

1. CTXΦ phage is a virus of filamentous morphology able to infect specifically the bacterium Vibrio cholerae without killing the cells.
2. Cholera toxin is a protein synthesized and released by Vibrio cholerae over the intestinal epithelium; enzyme activity of this protein inside the epithelial cells of the human intestine produces the clinical symptoms of cholera.
3. CTXΦ-encoded toxin genes are the cholera toxin genes (ctxAB), the zonula occludens toxin gene (zot) and the accessory cholera enterotoxin gene (ace).
4. Non-toxigenic Vibrio cholerae is any strain of this species devoid of the CTXΦ-encoded genes.
5. Vaccine is a medicinal preparation composed of a pharmaceutically active ingredient plus a vehicle and excipients.
6. Side effect of moderate intensity is a reaction induced in subjects receiving a vaccine, the reaction being able to limit but unable to impair daily activities of the subject.
7. Side effect of severe intensity is a reaction induced in subjects receiving a vaccine, the reaction being able to impair daily activities of the subject.
8. Safe vaccine is a medicinal preparation that does not induce side effects of severe intensity.
9. Hemagglutinin/protease is a protein secreted by Vibrio cholerae which has dual function, being one the ability to agglutinate erythrocytes and the other a proteolytic enzyme activity.
10. celA is the nucleotide sequence for the gene coding for a protein termed endoglucanase A. This protein naturally occurs in Clostridium thermocellum strains and has a β(1-4) glucan-glucane hydrolytic activity able to degrade cellulose and its derivatives.

Clinical cholera is an acute diarrheal disease characterized by the loss of valuable electrolytes and water. Diarrhea results from the action of cholera toxin on the intestinal epithelium. Cholera toxin is a protein produced by V. cholerae during infection of the human intestine. In up to 50% of the non-treated cholera cases the patient may die due to dehydration. An effective and safe cholera vaccine is still elusive despite more than 100 years of research.

Seven cholera pandemics have been recorded; the former six were caused by strains of serogroup O1 and classical biotype. In the current seventh pandemic V. cholerae of serogroup O1 and biotype El Tor predominate. In January 1991, the 7^{th} pandemic extended to South America causing more than 25 000 cases and over 2000 deaths in Peru, Ecuador, Colombia and Chile. By November 1992, a new serogroup with epidemic potential emerged in India and Bangladesh, the O139, which became soon a cause of concern in the developing world. These experiences reinforced the need for effective cholera vaccines against V. cholerae of serogroups O1 (El Tor biotype) and O139.

Convalescence to cholera is followed by protective immunity to the illness for at least 3 years. Many efforts done in Vibrio cholerae vaccinology are directed to obtain live cholera vaccines from strains attenuated in virulence. These vaccines when given by oral route should mimic the infection but do not produce disease or moderate or severe side effects.

Virulence attenuation involves deletion of toxin genes encoded by the CTXΦ phage. See patents of Kaper J. et al.; WO 91,18979 and Mekalanos J.; WO 95,18633.

Oral vaccination strategies date from 1970-1980 at the Center for Vaccine Development of Maryland, USA, using chemically mutagenized vibrios as pharmaceutically active ingredients. Reversion to virulence of these mutants impeded further spread of the strategy (Levine et al., Infect and 1 mm, No. 2, 1984; Finkelstein et al., patent U.S. Pat. No. 4,328,209) and prompted researchers to generate non-toxigenic mutants by precise genetic and non-reversible deletions. These mutants confer solid immunological protection against disease (Kaper J. B. and Levine M. Patents U.S. Pat. Nos. 06,472,276 and 581,406). However, their essential drawback is the high level of moderate and severe effects they produce in the vaccinees (Levine et al., Infect. and 1 mm. Vol 56, No. 1, 1988). Thus, one major issue to overcome in cholera vaccine development is reactogenicity.

Cholera researchers are concerned with the horizontal transfer of genetic information by bacteriophages or other genetically mobile elements among bacteria (Campos et al, J. Bacteriology 184 (24): 7231-7240). Thus, a second major issue to overcome when developing live cholera vaccines is reversion to virulence mediated by phages.

Dukoral is a cholera vaccine available at the market in which the pharmaceutically active ingredient is a mixture of inactivated whole cells of V. cholerae, supplemented with the B subunit of cholera toxin (Holmgren et al., Current topics in Microbiology and Immunology, Vol. 146, 1989). Dukoral is given by oral route, is safe and effective but requires multiple doses to generate an immune response equivalent to that of a cholera infection and consequently is expensive. Dukoral is not protective in children.

Orochol is an alternative live cholera vaccine available in the market. The pharmaceutically active ingredient of this vaccine is CVD103HgR, an attenuated strain of Vibrio cholerae of classical biotype (see patent USA, U.S. Pat. No. 5,399,494). Orochol is safe, effective and cheap in phase IIB studies (Tacket et al., Infect and Immun. 67(12): 6341-6345). However, the protective efficacy of Orochol in phase III trials is variable (Richie et al., Vaccine 18 (22): 2399-2410; Calain et al., Vaccine 22(2004): 2444-2451).

Patent WO 95/18633 describes additional live vaccine candidates of all serotypes of V. cholerae circulating in the current pandemic and the 0139 serotype. They are safe, their production is cheap and preliminary effective in phase IIB trials; however their efficacies need to be tested in phase III trials.

Robert et al., Vaccine, Vol. 14, No. 16, 1517-22, 1996, demonstrated the feasibility of interrupting the hemagglutinin/protease gene of Vibrio cholerae with a Clostridium thermocellum DNA fragment without impairing the colonizing ability of the vibrios. Colonization of the human small bowel by Vibrio cholerae is essential to induce a strong localized immune response of secreted IgA in the intestinal mucosa and to produce a long lasting immunity against cholera (Taylor et al., The Journal of Infectious Diseases, 1994, 170: 1518-23).

Medicinal preparations used as vaccines usually contain a pharmaceutically active ingredient admixed with excipients in a particular formulation. Thus, medicinal preparations are part of the vaccine kit and are admixed with the appropriate vehicle and given to people. Live vaccines are special in that the viable status of the active ingredient (the vaccine strain), need to be preserved during storage. Freeze drying is the technology of choice to prepare live bacterial vaccines.

However, freeze drying is a stressing process during which live bacteria loose viability if they are not adequately protected by the addition of certain substances termed lyoprotectants. The choice of lyoprotectans is specific for each bacterial species or strain and general recipes are not available. The lyoprotectants used will determine the final composition of the medicinal preparation as well as the specific parameters at the freeze drying process. No references are made in published papers to the exact composition of lyoprotectans used to prepare freeze dryed cholera vaccine strains.

### DESCRIPTION OF THE INVENTION

This invention discloses the methods to obtain and the composition of a medicinal preparation to vaccinate against cholera. It primarily discloses a method to construct attenuated strains to be used as pharmaceutically active ingredients of the vaccine. Due to recognized characteristics, strains constructed by this method are unable to induce moderate or severe effects in human subjects that ingest it. Vaccine strains are mutants of V. cholerae in which the hemagglutinin/protease gene (hap) is interrupted with a Clostridium thermocellum fragment of DNA encoding the celA gene. These mutants are constructed over a background of a non-toxigenic strain of V. cholerae.

The Hemagglutinin/protease (Hap) is the major secreted, mucin-degrading protease in strain C7258 and C6706 (Silva et al., Microbiology 149: 1883-1891). Hap is a putative virulence factor that may be involved in several pathogenic activities (Silva and Benitez, J. Bacteriol. 186(19):6374-6382).

Examples of non-toxigenic strains of V. cholerae useful to construct mutants for the present invention have been disclosed elsewhere (Benitez et al., Archives of Medical Research27: 275-283). More preferably, but not limited to, the mutants may be constructed from the non-toxigenic V. cholerae strains: 81, 413 or SG251 a (Table 1).

Vibrio cholerae strains 81, 413 and SG251a are characterized by the absence of all CTXΦ phage sequences in their chromosome, and by the presence of a single copy of the satellite RS1Φ phage. The nucleotide sequence of RS1Φ in 81, 413 and SG251a was confirmed by DNA sequencing. The methods of producing non-toxigenic mutants of V. cholerae are well described elsewhere (Archives of Medical Research, 27, No. 3, pp. 275-283).

TABLE 1
Strains that may be used to construct HA/P defective mutants

| Vaccine candidate | Biotype/Serotype | Genotype |
|---|---|---|
| 81 | EI Tor/Ogawa | ΔCTXΦ |
| 413 | EI Tor/Inaba | ΔCTXΦ |
| SG25-1a | O139 | ΔCTXΦ |

All strains and the methods of making are described in Archives of Medical Research, Vol. 27, No. 3, pp. 275-283, 1996. 81 and 413 derive from C7258 and C6706, respectively; both of which are clinical isolates from Peru, 1991.
SG25-1a is a derivative of the O139 isolate SG25-1 from Calcutta, India, 1993.

The invention also discloses a mixture of lyoprotectants that may be used to preserve viability of the constructed V. cholerae vaccine strains during the freeze drying process. The effective concentration of each substance in the mixture is disclosed. This methodology may be used to attain medicinal preparations to immunize against cholera as may be inferred from the examples provided in this patent application.

In summary, this invention discloses how to obtain the pharmaceutically active ingredient (PAI) of a medicinal preparation suitable to prevent cholera. The patent application also discloses important properties of the PAI and provides the means to attain and the composition of a medicinal preparation that may be used to induce immunological protection against the disease. The following examples present the data in more detail.

### EXAMPLES OF THE INVENTION

### Example 1

### Constructing the Pharmaceutically Active Ingredient of the Vaccine, the Attenuated Strains

Non-toxigenic strains of V. cholerae are only partially attenuated in virulence and are still inductive of moderate to severe side effects in subjects that ingest them. Non-toxigenic strains are thus non useful as pharmaceutically active ingredients (PAI) for live oral cholera vaccines. To construct useful strains, further attenuation needs to be attained.

Pathogenic functions attributed to the hemagglutinin/protease of V. cholerae, make the gene coding for this enzyme an eligible target for inactivation to construct strains intended to be used as PAI in oral cholera vaccines. Thus, a strategy was devised for the construction of hemagglutinin protease defective mutants to be used as the PAI in the medicinal preparation of the present invention.

Tagging of the attenuated strains with a marker gene that allow their distinction from other environmental isolates of V. cholerae is FDA required (Kaper et al. in New and improved vaccines against cholera 2^{nd} edition, chapter 33; editors M M Levine, G C Woodrow, J B Kaper and G S Gobon, Marcel Dekker, Inc, New York). The Clostridium thermocellum endoglucanasa A gene codes for a protein able to degrade cellulose. This enzyme activity is easy to detect in bacterial colonies grown on agar plates overlaid with a carboxymethylcellulose-agar indicator gel followed by incubation for 2 hours at 72° C., Congo red staining and washing with 1M NaCl. Thus, the gene coding for this enzyme was among the genes that might be selected as the marker gene.

The strategy devised was aimed at replacing the natural hemagglutinin protease gene of V. cholerae with a mutant allele interrupted by a Clostridium thermocellum DNA piece coding for the endoglucanase A gene (celA). The recombinant allele of hap was first constructed in vitro. Plasmid pCH2 (Hase and Finkelstein, J. Bacteriol. 173(11):3311-3317) is a derivative of pACYC184 containing a Hind III DNA fragment from V. cholerae coding for the hemagglutinin protease gene. pCH2 was cut by the unique StuI site present in the hap coding sequence. In parallel, the HindIII DNA fragment of Clostridium thermocellum contained in plasmid pCT104 (Beguin et al., J. Bact. 162: 102-105) was excised, blunted with klenow and cloned into the StuI-linearized pCH2 plasmid vector. The resultant plasmid was termed pACH3.

The hap::celA allele was excised with HindIII from pAHC3 and cloned into HindIII digested pIJ2925 (Jansen and Bibb, Gene 124: 133-134) to obtain plasmid pIJHC1. In this new construct, the hap allele is between two BglII restriction enzyme sites. Thus, pIJHC1 was digested with BglII and the fragment containing the hap allele was cloned into the unique BgIll restriction site of the suicide vector pGP704 (Miller and Mekalanos, J. Bacteriol. 170(6):2575-2583). The resultant suicide plasmid was denominated pGPH6 (FIG. 2) and was used to transform the donor Escherichia coli strain SM10λpir.

Non-toxigenic strains indicated in Table 1 were chosen to construct hemagglutinin/protease defective mutants. All three strains were equally and independently processed. Plasmid pGPH6 was transferred from E. coli SM10λpir to the non-toxigenic mutant of V. cholerae to produce an ampicillin resistant strain, termed co-integrate, due to integration of pGPH6 into homologous sequences within the bacterial chromosome.

Southern hybridization experiments of XhoI-digested DNAs probed with a hap-specific labeled-probe demonstrated that in all clones analyzed a single restriction fragment of 21 kb hybridized with the hap-specific probe. This indicated that integration of pGPH6 in all clones occurred specifically directed to the hap allele.

Then, the above ampicillin-resistant co-integrate was allowed to segregate by ten passes in antibiotic-free broth and tested for ampicillin-resistance. Some ampicillin-sensitive colonies were recovered and characterized by Southern blot with the hap specific probe.

Screening by Southern blot performed on XhoI digested DNA of the ampicillin-sensitive clones arisen, allowed the identification of several strains having a single XhoI fragment of 9.5 kb that hybridized with the probe. This absolutely evidenced that in these clones the natural allele of hap became replaced by the interrupted allele. Clones derived from strains 81, 413 and SG251a were denominated 638, 1333 and L911, respectively.

### Example 2

### Non-clinical Evaluation of Functional Properties of the Attenuated Strains

Infant Mouse Colonization Assay

The infant mouse colonization assay (Herrington et al, J. Exper. Med. 168:1487:1492, 1988) was used to asses the colonization properties of each mutant. An inoculum of 10⁵-10⁶ vibrios in a final volume of 50 μl was administered intragastrically to groups of at least five mice. After 18-24 hours at 30° C., mice were sacrificed; the intestine was dissected, homogenized and plated on bacteriological media containing appropriate supplements to support growth of mutants. Colonies that grew after overnight incubation were identity checked by the enzyme activity of the endoglucanase A. According to the results shown in Table 2 all strain colonized the suckling mice intestine. It is concluded that all strains are functional in colonization.

TABLE 2
Colonizing capacities of hap::celA cholera vaccine strains

| | | | Biotype/ | |
|---|---|---|---|---|
| Strain | Input | Output | Serotype | Relevant genotype |
| 638 | 1.0 × 10⁶ | 2.8 × 10⁵ | EI Tor/Ogawa | Δctxφ, hap::celA |
| 1333 | 2.0 × 10⁶ | 4.2 × 10⁵ | EI Tor/Inaba | Δctxφ, hap::celA |
| L911 | 1.2 × 10⁶ | 8.0 × 10³ | O139 | Δctxφ, hap::celA |
| 638T | 1.7 × 10⁶ | 6.0 × 10⁵ | EI Tor/Ogawa | Δctxφ, hap::celA, thyA⁻ |

Detection of Protease Activity on Milk Agar Plates

Milk-LB plates were used to detect proteolytic activities in supernatants of TSB-grown vibrios. For quantification of protease activity, the azocasein method was adapted from Ginther, Antimicrob. Agents Chemother. 15, 522-526, 1979. Briefly, 1.1 ml of buffer (CaCl₂ 1 mM; Tris 0.2M, pH 7.2; Azocasein 1%); were mixed with 200 μl of culture supernatant and incubated for 1 hr at 37° C. The unreacted substrate was precipitated with 83 μl of TCA 40% for 10 min. followed by 10 min. centrifugation at 12000 rpm. The colored product remaining in solution was neutralized with NaOH and read at 450 nm. One unit of enzymatic activity was defined as the quantity of enzyme producing a net increase of one in the optical density of the sample in one hour of reaction.

The replacement of the hemagglutinin/protease gene by the allele interrupted with the DNA fragment from Clostridium thermocellum, generated in the strains disclosed herein a reduction in 60-80% of the proteolytic activity when compared to their non-toxigenic parents.

Detection Expression of the Endoglucanase A Marker Gene in Colonies of V. cholerae

Detection of the endoglucanase A activity was done on colonies of V. cholerae grown on LB plates for 24 hours at 37° C. The colonies were overlaid with a thin coat of 0.3% carboxymethyl-cellulose, 0.3% agarose—gel, which was prepared in phosphate—citrate buffer, pH 6.3. The plates were incubated for 4 hours at 72° C. and endoglucanase A positive colonies were visualized by staining with a 1% Congo Red solution and washing with 1M NaCl. Enzyme activity was declared positive for such colonies seen as red colonies surrounded by a transparent halo in the red background of the plate.

The celA marker used to unequivocally distinguish the vaccine, was found to be stably expressed and inherited in V. cholerae. The appearance of tagged vibrios can be observed in FIG. 1.

Assay for Motility

Cells from single and well isolated colonies were picked from a master LB plate aided with the tip of a platinum needle and inoculated by insertion (2-3 mm) into a motility agar plate (LB, agar 0.4%). The diameter each colony spread through soft agar was recorded at 24 hours of incubation at 30° C. A bacterial strain was considered non-motile if it spread 3 mm or less from the point of application. A bacterial strain spreading >3 mm beyond the point of inoculation was considered motile.

All strains under the scope of this patent, but L911 were found to be motile. L911 is a derivative of SG251a, which in our hands, resulted non-motile when assayed in motility agar.

### Example 3

### Phase I Studies with Freshly Grown Strain 638

Growth and Harvesting of the PAI

Strain 638 was cultured on Brain Heart Infusion (Oxoid, CM55) agar plates for 5 hours at 37° C. Grown bacteria was scrapped from agar surface and suspended in 0.9% sterile saline.

Dosage

A single oral dose of live vaccine was prepared at potencies of 10⁷, 10⁸ and 10⁹ viable cells per immunizing dose. Each dose was administered to people suspended in 30 ml of 2% sodium bicarbonate buffer.

Clinical Testing of Strain 638

Freshly grown strain 638 was given to 42 volunteers aged 18-40. A summary of clinical findings after ingestion of strain 638 and placebo is shown in Table 3. All clinical manifestations observed were mild and of short duration. No statistical significance could be demonstrated between the vaccine and placebo groups with the present data. Gurgling and abdominal cramps were the reactions more frequently reported by volunteers irrespective of dose. Four volunteers developed mild diarrhea (grade 3). Three of these volunteers received the high dose and one the medium dose. One volunteer, who received the high dose, had 5 loose stools (72 h after inoculation) and a total output of 680 g. Two volunteers had 2 loose stools 28 and 72 h after inoculation with total outputs of 220 and 500 g, respectively. The other volunteer had a single diarrheal output of 300 g at 73 h after inoculation.

Bacteriological Isolation of Vaccine Strain

As indicated in Table 4, strain 638 was recovered from 37 out of 42 volunteers inoculated (88%). For the higher dose, excretion of the vaccine strain tend to peak at 72 h after inoculation. Three out of the 4 cases of diarrhea occurred at this time. Of the 37 volunteers that excreted vibrios, 12 excreted on at least 4 days, 19 on at least 3 days, and 28 on 2 days. The number of volunteers excreting strain 638 and the mean number of vibrios/g stool declined in the lower dose. Vibrios isolated from the stool of volunteers produced endoglucanase A indicating that the celA reporter gene was stably maintained during growth in the human intestine. We conclude that strain 638 is a good colonizer of the human small bowel.

Immune Response to Vaccine Strain.

Strain 638 elicited a significant and consistent immune response in terms of serum vibriocidal antibodies, serum anti-Ogawa LPS IgG or IgA, and Ogawa LPS-specific IgA ASC (Tables 5 y 6). Although reciprocal GMT peaked 14 days after inoculation, seroconversion was attained on day 7 and titers remained high until day 28. Seroconversion rates, peak reciprocal GMT, and ELISA titers were dose-dependent. However, even at the lowest dose, strain 638 elicited a significant vibriocidal antibody response compared to placebo. A significant proportion of the volunteers which experimented seroconversion developed relatively high (≧1024) vibriocidal titers (Table 6). The high percentage of responders in the ASC evaluation (Table 5) reflects an effective stimulation of mucosal immunity, mainly sigA, by strain 638 in correspondence with the elevated anti-LPS IgA titers encountered 14 days after inoculation. One volunteer who ingested placebo seroconverted for anti-LPS IgG. This volunteer had very low pre-inoculation anti-LPS serum IgG which increased to the cutoff value at day 7 and remained constant thereafter. Another volunteer who ingested placebo reached the cutoff value of ASC. Similarly, this volunteer had a very low pre-inoculation number of LPS-specific ASC. We conclude that strain 638 elicits a significant immune response.

TABLE 3
Frequency of occurrence of adverse reactions after ingestion
of EI Tor Ogawa candidate vaccine strain 638.

| | Group of volunteers | | | | | | Prob- |
|---|---|---|---|---|---|---|---|
| | Inoculated¹ | | Placebo² | | | Confidence | ability |
| Symptom | + | − | + | − | R.R.³ | interval⁴ | (Fisher) |
| Diarrhea | 4 | 38 | 1 | 13 | 1.33 | 0.16-10.96 | 0.6329 |
| Abdominal | 13 | 29 | 2 | 12 | 2.17 | 0.56-8.44 | 0.1944 |
| cramps | | | | | | | |
| Gurgling | 14 | 28 | 3 | 11 | 1.56 | 0.52-4.63 | 0.3143 |
| Heartburn | 6 | 36 | 2 | 12 | 1.00 | 0.23-4.40 | 0.6850 |
| Headache | 7 | 35 | 0 | 14 | — | — | 0.1163 |
| Vomiting | 1 | 41 | 0 | 14 | — | — | 0.7500 |

Volunteers with diarrhea

| | |
|---|---|
| Mean diarrheal stool weight (range) | 425 g (220-680) |
| Mean number of diarrheal stools per | 2 (1-5) |
| ill volunteer (range) | |

Notes:
¹N = 42,
²N = 14,
³Relative Risk,
⁴(95%).

TABLE 4
Recovery of Vibrio cholerae strain 638 from the stools of volunteers.

| | Volunteers excreting vaccine strain/total | | | | | | |
|---|---|---|---|---|---|---|---|
| Group of | Time after inoculation (h) | | | | | | Mean |
| Volunteers | 24 | 48 | 72 | 96 | 120 | Total | CFU/g stool |
| High dose | 7/29 | 10/29 | 16/29 | 15/29 | 10/29 | 28/29 | 4.4 × 10⁶ |
| (1-2 × 10⁹) | | | | | | | |
| Medium dose | 6/6 | 5/6 | 4/6 | 4/6 | 3/6 | 6/6 | 5.5 × 10⁶ |
| (2 × 10⁸) | | | | | | | |
| Low dose | 1/7 | 2/7 | 2/7 | 2/7 | 2/7 | 3/7 | 2.7 × 10⁵ |
| (4 × 10⁷) | | | | | | | |

TABLE 5
Anti-LPS IgA ASC response in peripheral blood of volunteers
following ingestion of Vibrio cholerae strain 638

| | | Mean ASC per |
|---|---|---|
| Dose | Positives (%) | 10⁷ PBMC (range) |
| High | 27/29 (93.1) | 485 (0-4750) |
| Medium | 6/6 (100) | 377 (40-1285) |
| Low | 6/7 (85.7) | 5 (0-65) |
| Placebo | 1/14 (7.1) | 371 (0-2040) |

TABLE 6
Serum antibody responses in volunteers orally administered
Vibrio cholerae EI Tor Ogawa strain 638

| | Group of volunteers | | | |
|---|---|---|---|---|
| Response | High dose | Medium dose | Low dose | Placebo |

Vibriocidal antibodies

| | | | | |
|---|---|---|---|---|
| Seroconversion | 24/29 (82) | 5/6 (83) | 5/7 (71) | 0/14 (0) |
| rate¹ (%) | | | | |
| GMT (range): | | | | |
| Pre-inoculation | 47 (0-160) | 32 (0-40) | 33 (0-40) | 37 (0-320) |
| Post-inoculation | 873 | 639 | 389 | 46 |
| peak [14 days] | (0-20480) | (0-2560) | (40-2560) | (0-320) |
| Responders with | 10/24 | 4/5 | 4/5 | 0 |
| titers ≧ 1024 | | | | |

Anti-Ogawa LPS IgG

| | | | | |
|---|---|---|---|---|
| Seroconversion | 23/29 (79) | 4/6 (67) | 3/7 (44) | 1/14 (7) |
| rate² (%) | | | | |
| Log reciprocal titer³ ± SD: | | | | |
| Pre-inoculation | 0.12 ± 0.25 | 0.12 ± 0.29 | 0 | 0.03 ± 0.12 |
| Post-inoculation | 1.86 ± 1.12 | 1.59 ± 1.49 | 1.07 ± 1.36 | 0.19 ± 0.57 |
| Peak [14 days] | | | | |

Anti-Ogawa LPS IgA

| | | | | |
|---|---|---|---|---|
| Seroconversion | 26/29 (90) | 6/6 (100) | 5/7 (71) | 0/14 (0) |
| rate² (%) | | | | |
| Log reciprocal titer³ ± SD: | | | | |
| Pre-inoculation | 0.1 ± 0.37 | 0 | 0.27 ± 0.37 | 0.13 ± 0.39 |
| Post-inoculation | 2.43 ± 1.0 | 2.68 ± 0.48 | 1.96 ± 1.25 | 0.19 ± 0.53 |
| peak [14 days] | | | | |

Notes:
¹Number of volunteers with fourfold increase in titer/total,
²Number of volunteers with a twofold increase in titer/total,
³logarithm of the reciprocal arithmetic mean titer.
Abbreviations:
GMT, geometric mean titer;
SD, standard deviation of the mean.

Live Vaccine Strains

Strains 638, 1333 and L911 herein described can be used to induce immunological protection against cholerae in humans, provided the high rates of seroconversion they elicit and the low levels of adverse reactions they produce when orally administered to volunteers. Depending upon relevant local epidemiology, a single strain or their combinations of them could be used for immunization.

### Example 4

### Methods to Produce Medicinal Preparations Suitable for Oral Administration to Humans to Immunize Against Cholera

Culturing the Vaccine Strains

The microorganisms were cultured in 500 ml of LB broth (NaCl, 1%; triptone, 1%; yeast extract, 0.5%) at 37° C. with aeration in a New Brunswick Scientific orbital shaker under agitation of 150 to 250 cycles per minute until reaching the logarithmic phase (1.0 unit of optical density at 600 nm). Cells were harvested by centrifugation at 5000×g and 4° C. during 15 minutes.

Admixing with Lyoprotectants

Cell pellets were resuspended in 100 ml of a sterile dissolution containing 6 g of skim milk, 2 g of sorbitol and 2 g of peptone, so that the cellular concentration was about 10⁹ cells ml⁻¹. Two ml of the suspension were dispensed into 10R type glass vials and immediately subjected to deep freezing at −60° C. for 12 hours.

Freeze Drying Process

Primary drying was conducted for 12 hours at −33° C. Secondary drying was conducted at 20° C. for 14 hours hours. After freeze drying, vials were closed with rubber caps under vacuum and sealed with a pull-off aluminum cap to avoid vacuum release and viability losses. Viable losses were calculated as the rate of the CFU log before and after carrying the freeze drying process, or before and after storage of the lyophilized material.

Results of the Freeze Drying Process

The lyophilized material of strain 638 dissolved instantly in 2.0% sodium bicarbonate solution. Post lyophilization counts of Vibrio cholerae in the vials were between 1 and 5×10⁹ CFU. Pre- to post freeze drying viability loss were calculated in 0.48, 0.52 and 0.55 logarithmic orders, with a lower limit of 0.1.

Effect of Humidity and Oxygen on the Medicinal Preparation

Samples were opened and stored for 3 days at 25° C. in an atmosphere of a sterile glass desiccator, under 11% relative humidity, created by a solution of saturated lithium chloride. Viability loses in the preparation resulted to be 1.6 logarithmic orders, indicating that vacuum and humidity are important factors that need to be controlled in the medicinal preparation.

Effect of the Temperature of Storage

The medicinal preparation containing strain 638 was stored for 1 year either at 8° C. or −20° C. Table 7 shows the calculated viability losses obtained.

TABLE 7
Viability loss for 1 year of storage

| Strain | 8° C. | −20° C. |
|---|---|---|
| 638 | 1.02 | 0.59 |

### Example 5

### Phase I Study to Determine Immunogenicity of the Medicinal Preparation

Study Desing

A total of 13 healthy volunteers were recruited and admitted to the isolation ward for biological risks at the Institute for Tropical Medicine in Havana Cuba. Volunteers were acclimated to the isolation ward for 24 hours. The following day, fasted volunteers were given 100 ml of 2% sodium bicarbonate solution to neutralize stomach acidity; the solution of sodium bicarbonate being prepared in mineral pure water. Thirty minutes later, one group (n=9 volunteers) received one vaccine dose and another group (n=4) received the placebo.

Vaccine Preparation

The content in each vial, containing a potency of 1-5×10⁹ viable bacteria of PAI (the vaccine strain 638) was dissolved in 30 ml of 2% sodium bicarbonate buffer prepared in a solution of pure mineral water and ingested by the volunteer.

Placebo

The placebo consisted of 30 ml of a 2% sodium bicarbonate solution prepared in pure mineral water.

Bacteriological Isolation of Vaccine Strain

Strain 638 was recovered from the feces of 8 (89%) out of 9 volunteers inoculated but from none of the placebo recipients. Peak excretion was achieved by 96 hours. The median incubation period to excretion had a value of 1 day with lower (LQ) and upper (UQ) quartiles at 1 and 3 days, respectively. Fecal shedding lasted for a median of 5 days (LQ=3 days; UQ=5 days). Vibrios isolated from the stools of volunteers produced endoglucanase A indicating that the celA reporter gene was stably maintained during laboratory growth and during obtainment and storage of the medicinal preparation. We conclude that strain 638 is acceptable as PAI for a medicinal preparation against cholera because it keeps its colonization properties during the process.

TABLE 8
Fecal shedding of strain 638 by volunteers ingesting
the medicinal preparation

| | CFU of strain 638 shed by gram of feces in | | | | |
|---|---|---|---|---|---|
| | volunteers receiving a single oral dose | | | | |
| | of the medicinal preparation. | | | | |
| Parameter | 24 h | 48 h | 72 h | 96 h | 120 h |
| Geometric | 1.1.E+05 | 1.9.E+05 | 3.4.E+05 | 4.5.E+06 | 2.3.E+04 |
| mean | | | | | |
| Max. | 5.0.E+07 | 1.0.E+10 | 1.0.E+07 | 3.0.E+10 | 2.0.E+06 |
| Min. | 1.0.E+02^{(a)} | 1.0.E+02 | 1.0.E+02 | 1.0.E+02 | 1.0.E+02 |

^{(a)}The lower limit in fecal shedding detection is 10² CFU per gram of stool.

Results of the Functional Bactericidal Assay to Evaluate the Immune Response to the Medicinal Preparation

The functional assay for bactericidal antibodies is the best surrogate marker of protection against cholera in healthy volunteers exposed by first time to a vaccine preparation. Eight among nine volunteers (89%) who received the medicinal preparation in the vaccine group seroconverted with antibodies with functional complement dependent bactericidal activity against Vibrio cholerae. None of the placebo recipients seroconverted. Titers of bactericidal activity peaked at day 14 (Table 9). In correspondence with these results, we interpret that a medicinal preparation obtained by the procedures described in the present invention is able to induce an immune response against Vibrio cholerae. In terms of magnitude, these titers may be expected to afford protection against cholera.

TABLE 9
Immune response induced in volunteers by a single oral dose of a medicinal
preparation to immunize against cholera containing the strain 638 as the PAI.

| | Geometric mean titer (range) of systemic bactericidal antibodies | | | | |
|---|---|---|---|---|---|
| | induced by the medicinal preparation, measured at the | | | | |
| | following sampling times: | | | | |
| Group | Pre-vaccination | 7 days | 14 days | 21 days | 28 days |
| Vaccine | 37 (20-80) | 941 (80-5120) | 1881 (20-10240) | 1280 (80-5120) | 1280 (640-2560) |
| Placebo | 34 (20-40) | 57 (20-80) | 57 (20-80) | 24 (10-80) | 28 (10-80) |

The limit of detection of the assay for functional bactericidal antibodies was set at the titer of 1:20.

From the results presented in the present section one may conclude that the combination of lyoprotectants, the buffer and the freeze dried strains constitute a medicinal preparation suitable for administration to humans to induce immunological protection against cholera.

### BRIEF DESCRIPTION OF THE DRAWINGS

FIG. 1. Detection of the endoglucanase activity associated to agar grown colonies of V. cholerae tagged with the ce/A marker gene in assays with CMC-indicator agar.

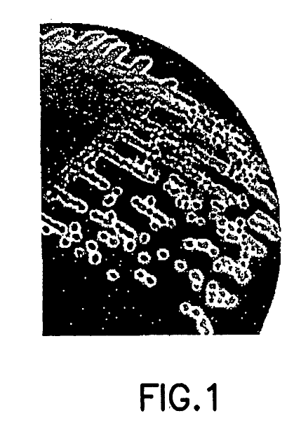

FIG. 2. Schematic representation of suicide vector pGPH6 used to replace the hap gene of V. cholerae by the recombinant hap::celA allele.

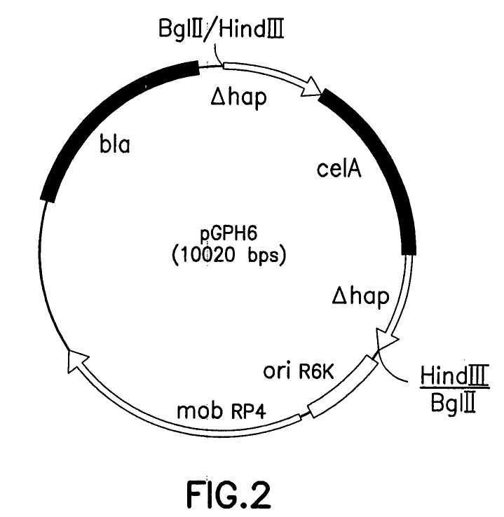

### ADVANTAGES

This invention provides us with an approach to create mutants of Vibrio cholerae unable to produce moderate or severe side effects in subjects ingesting them, the method consisting in inactivation of the hemagglutinin/protease gene of non-toxigenic strains.

Such new mutants are useful as the PAI of medicinal preparations aimed at inducing immunological protection against cholera by oral route. These mutants may be used in combination in a medicinal preparation to prevent disease due to O1 and O139 Vibrio cholerae.

### Deposits

Strains constituting the subject matter of the present invention were deposited at the DSMZ under the collection numbers are listed below:

Vibrio cholerae 1333 (DSM 12757)

Vibrio cholerae L911 (DSM 12758)

Vibrio cholerae 638 (DSM 12759)

1. A medicinal preparation suitable for oral administration to humans for inducing immunological protection against cholera comprising as essential ingredients:
a) one or more freeze-dried Vibrio cholerae vaccine strains derived from a non-toxigenic parental, the strain or strains being characterized by an acceptable level of residual reactogenicity due to the presence of a dysfunctional hemagglutinin/protease gene (hap), resulting from deletion, insertion or any other defined and irreversible genetic manipulation.
b) a mixture of lyoprotectants consisting of 2% skim milk, 2% bacteriological peptone and 6% sorbitol,
c) a 2% sodium bicarbonate acid neutralizing buffer, and
d) an aqueous vehicle of comprising pure mineral water.

2. The preparation of claim 1 wherein the said dysfunction results from the insertion in the hap gene of the marker gene celA,

3. The preparation of claim 1, said vaccine strains comprising one or more vaccine strains selected from the following:
(i) a vaccine strain belonging to the El Tor Biotype of Vibrio cholerae;
(ii) a vaccine strain belonging to the serotypes Inaba or Ogawa of Vibrio cholerae Biotype El Tor; and
(iii) a vaccine strain belonging to the 0139 serogroup of Vibrio cholerae.

4. The preparation of claim 1 comprising one or more of the following vaccine strains deposited at DSMZ-Deutsche Sammlung von Mikrooganismen und Zellkulturen Gmbh:
Vibrio cholerae 1333 (DSM 12757);
Vibrio cholerae L911 (DSM 12758);
Vibrio cholerae 638 (DSM 12759).

5. A single oral administration dose medicinal preparation for immunological protection against cholera comprising:
a) a freeze-dried Vibrio cholorea vaccine strain derived from a non toxigenic parental strain, the vaccine strain having an effective level of reactogenicity for administration in a human, and further comprising an irreversibly genetically manipulated dysfunctional hemagglutinin/protease hap gene;
b) lyoprotectants;
c) a buffer; and
d) an aqueous vehicle;
whereby with the single oral administration of the medicinal preparation, there is immunological protection against cholorea.

6. The medicinal preparation of claim 1, wherein systemic bactericidal antibodies were present in the human from about 1 day to a peak in about 14 days after said single dose oral administration of the medicinal preparation.

7. The medicinal preparation of claim 1, said dysfunctional hemagglutinin/protease hap gene comprises an insertered marker gene celA.

8. The medicinal preparation of claim 5, said lyprotectants comprise skim milk, bacteriological peptone and sorbitol.

9. The medicinal preparation of claim 5, said buffer comprises sodium bicarbonate.

10. The medicinal preparation of claim 5, wherein the non-toxigenic strains are sufficiently attenuated so that the medicinal preparation is substantially free of adverse side effects.

11. The medicinal preparation of claim 5, wherein the medicinal preparation comprises from about 10⁷ to 10⁹ viable cells per said single dose, and whereby said immunization is effected.

12. The medicinal preparation of claim 11, wherein systemic bactericidal antibodies were present in the human from about 1 day to a peak in about 14 days after said single dose oral administration of the medicinal preparation, and wherein the non-toxigenic strains are sufficiently attenuated so that the medicinal preparation is substantially free of adverse side effects.

13, The medicinal preparation of claim 5, wherein the lyoprotectants are commensurate with the strain.

14. The medicinal preparation of claim 13, said lyoprotectants comprise skim milk, sorbitol and peptone.
